(19) United States
(12) Patent Application Publication
(10) Pub. No.: US 2026/0142019 A1
(43) Pub. Date: May 21, 2026

(54) METHODS AND SYSTEMS FOR PRIVACY-ENABLED CAMERA LIVE IMAGE CAPTURE

(71) Applicant: GE Precision Healthcare LLC, Waukesha, WI (US)

(72) Inventors: Dattesh Dayanand Shanbhag, Bengaluru (IN); Krishna Seetharam Shriram, Bengaluru (IN); Deepa Anand, Bengaluru (IN); Sajith Rajamani, Bengaluru (IN)

(21) Appl. No.: 18/951,490

(22) Filed: Nov. 18, 2024

(51) Int. Cl. G16H 30/20; G06F 21/62; G06V 40/16; G16H 30/40

(52) U.S. Cl. CPC G16H 30/20; G06F 21/6245; G06V 40/161; G16H 30/40

(57) ABSTRACT

Described herein are systems and methods that enable generation and storage of an accessible privacy preserved image that includes camera image data, obscures privacy regions, and is accessible via a standard medical imaging format. A method may include acquiring camera image data via a camera and acquiring scanner image data via a scanner, identifying a privacy region and obscuring camera image data in the privacy region to generate a privacy preserved camera image, and registering the privacy preserved camera image with scanner image data using a camera-scanner coordinate transformation matrix to generate an accessible privacy preserved image.

### TECHNICAL FIELD

Embodiments of the subject matter disclosed herein relate to methods and systems for image capture via an imaging system, and more specifically, to image modification to remove personally identifiable elements from an image.

### BACKGROUND

Camera systems with red, green, blue (RGB) sensors and depth information sensors are being increasingly leveraged for enabling efficient diagnostic imaging workflows, such as patient setup in diagnostic radiology exams including computed tomography (CT) and magnetic resonance (MR) systems. Use of these camera systems has reduced common errors in patient setup, and thereby increased throughput in diagnostic imaging. Typically, a live image of an imaging subject is shown on a display, and a technologist may view the live image to verify the patient setup. In traditional imaging setups involving scout and/or localizer images, a patient scan prescription setup is captured and stored in a standard medical imaging format so that technologists may easily view the imaging setup used to image the subject, and make decisions about an accuracy of the scan prescription and if the scan prescription is desired for use in a follow-up exam. For example, the patient scan prescription setup may be stored as a Digital Imaging and Communication in Medicine (DICOM®) image.

A similar imaging capture with camera-based patient setup that is compatible with a standard medical imaging format is therefore desirable to store relevant information, including landmarks and additional information specific to the imaging procedure (e.g., coil setup in MR systems). For example, the standard medical imaging format may be a DICOM® format. It may be desirable to access camera-based patient setup information via a DICOM® framework in such a way that real-world distances of the camera-base patient setup may be measured within the DICOM® framework. However, such a setup may be precluded due to patient privacy concerns, since images captured by RGB camera systems include information that can be used to easily identify the patient captured in the image, which may intrude into subject privacy. Methods may exist for camera-based patient capture, however images may not be saved, which may be due to privacy concerns. As the captured images are stored in an archival system, this would put the patient identity at risk in perpetuity.

### BRIEF DESCRIPTION

Described herein are systems and methods that enable generation and storage of an accessible privacy preserved image that includes camera image data, obscures privacy regions in the generated image, and is accessible via a standard medical imaging format. For example, a method for a medical imaging system may include acquiring camera image data captured via a camera and acquiring scanner image data captured via a scanner. The camera image data includes red, green, blue (RGB) image data and depth map image data. The method further includes identifying a privacy region in the camera image data and obscuring camera image data in the privacy region to generate a privacy preserved camera image. The privacy preserved camera image is registered with scanner image data using a camera-scanner coordinate transformation matrix to generate an accessible privacy preserved image that includes camera image data, obscures privacy regions in the generated image, and is accessible via a standard medical imaging format. The accessible privacy preserved image is stored in such a way that the accessible privacy preserved image can be viewed and navigated within a standard medical imaging format viewer.

In another example, the method includes capturing camera image data via a camera, identifying a privacy region in the camera image data via a landmark detection algorithm, applying a table-retention algorithm to the camera image data to remove camera image data from outside of a table region, obscuring camera image data in the privacy region to generate a privacy preserved camera image. The method further includes adding additional layers of information from a scanning procedure to the privacy preserved camera image. Scanner image data is captured via a scanner. The privacy preserved camera image is registered with scanner image data, using a camera-scanner coordinate transformation matrix, to generate an accessible privacy preserved image that includes camera image data, obscures privacy regions in the generated image, and is accessible via a standard medical imaging format. The privacy preserved camera-based patient setup information as the accessible privacy preserved image that can be viewed and navigated within a standard medical imaging format viewer.

The methods may be implemented by an imaging system, comprising a camera configured to capture camera image data, a scanner configured to capture diagnostic imaging data, a table configured to receive an imaging subject, and a computing device having instructions stored on non-transitory memory and executable by a processor. The instructions are executable by the processor to acquire camera image data of a field of view including the imaging subject and the table via the camera, execute a table retention algorithm to remove camera image data other than image data of the imaging subject and the table from a camera image, execute a landmark detection algorithm configured to identify a privacy preserving region of the imaging subject, obscure camera image data of the camera image in the privacy preserving region to obfuscate personally-identifying image data and generate a privacy preserved camera image, register the privacy preserved camera image with the diagnostic imaging data captured by the scanner a camera-scanner coordinate transformation matrix to generate an accessible privacy preserved image that includes camera workflow information, obfuscated personally-identifying image data obfuscated, and is accessible via a standard medical imaging format, and store the accessible privacy preserved image in a memory of the computing device.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows a pictorial view of an exemplary imaging system;

FIG. 2 shows a block schematic diagram of the exemplary imaging system;

FIG. 3 shows a set of images illustrating conventional image capture;

FIG. 4 shows an example setup of a camera configured for camera image data capture and an example camera image captured by the camera;

FIG. 5 shows example images of RGB image data and depth map image data included in the camera image data captured by the camera;

FIG. 6 shows example placement of a privacy region bounding box on RGB image data, and a privacy preserved camera image with camera image data obscured in the privacy region;

FIG. 7 shows example placement of user-selected and artificial intelligence (AI)-prescribed landmarks on depth map image data and the privacy preserved camera image;

FIG. 8 shows example images of the privacy preserved camera image and an accessible privacy preserved image;

FIG. 9 shows a set of images illustrating image capture for an accessible privacy preserved image;

FIG. 10 shows a flowchart of a method for generating an accessible privacy preserved image that includes information captured by a camera and by a diagnostic scanner;

FIG. 11 shows a flowchart of a method for a table retention algorithm that may be used in the method of FIG. 10 to identify a privacy region in the camera image data; and

FIG. 12 shows a block diagram of an exemplary imaging system.

### DETAILED DESCRIPTION

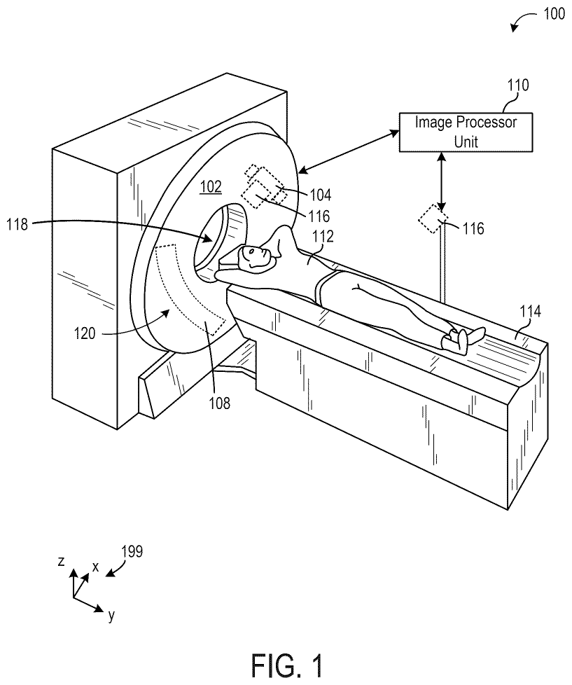

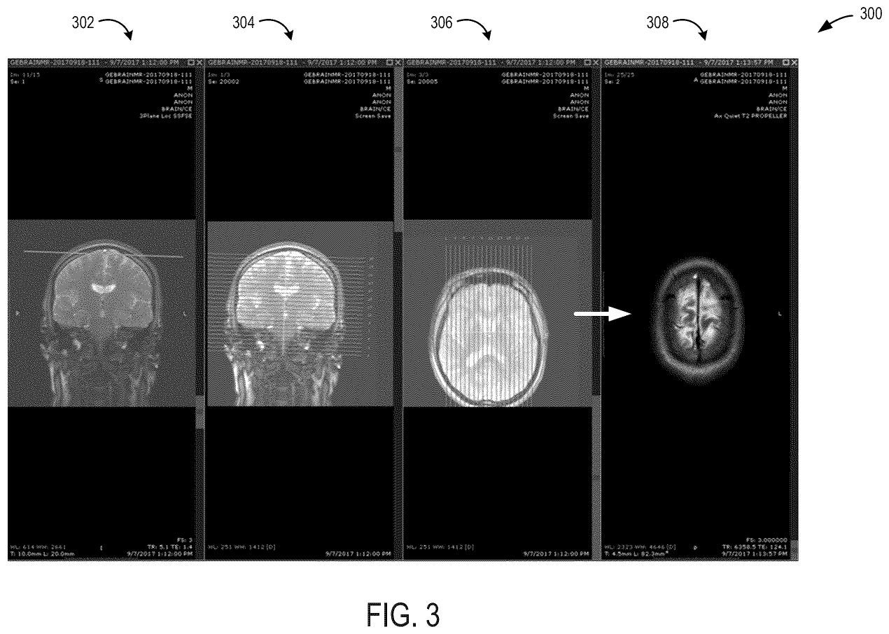

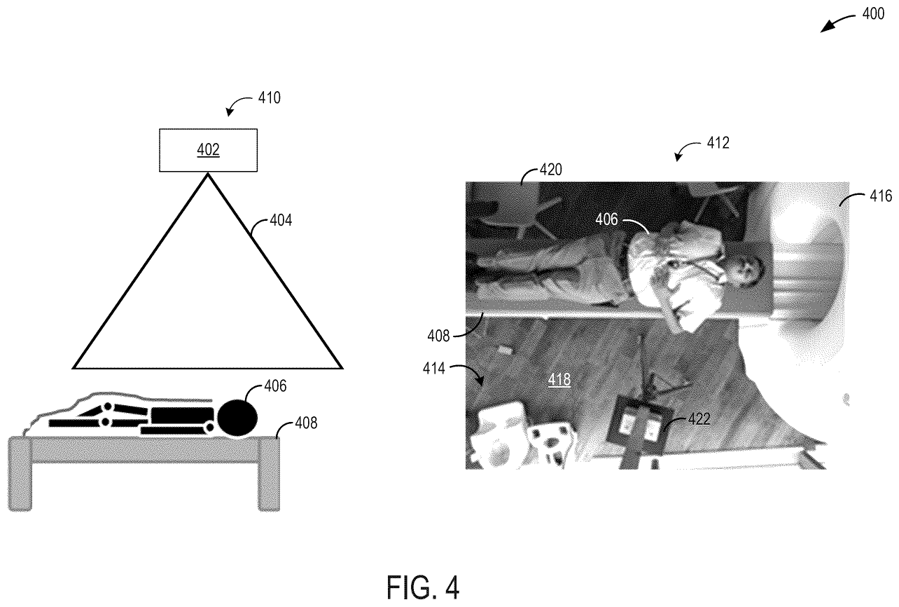

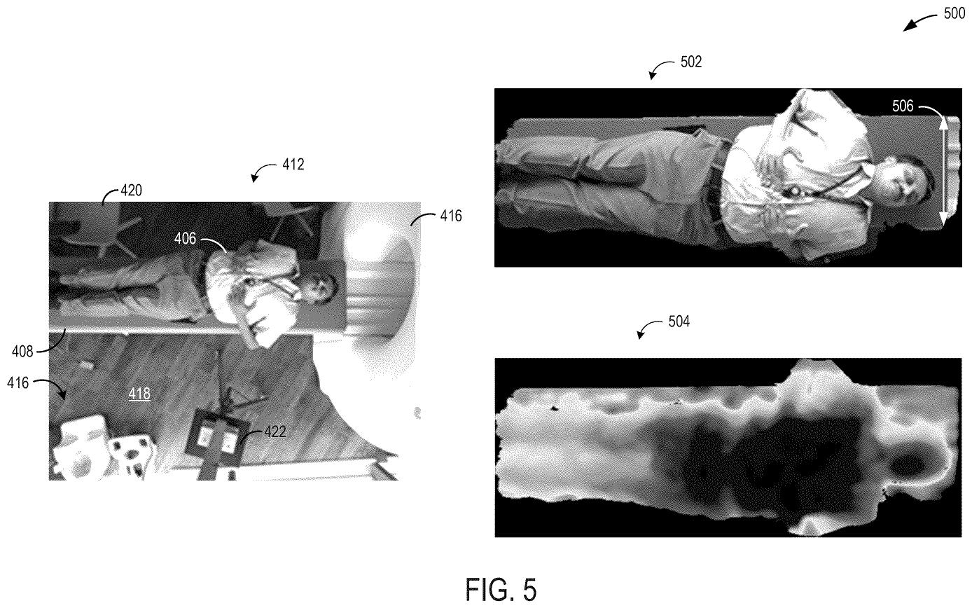

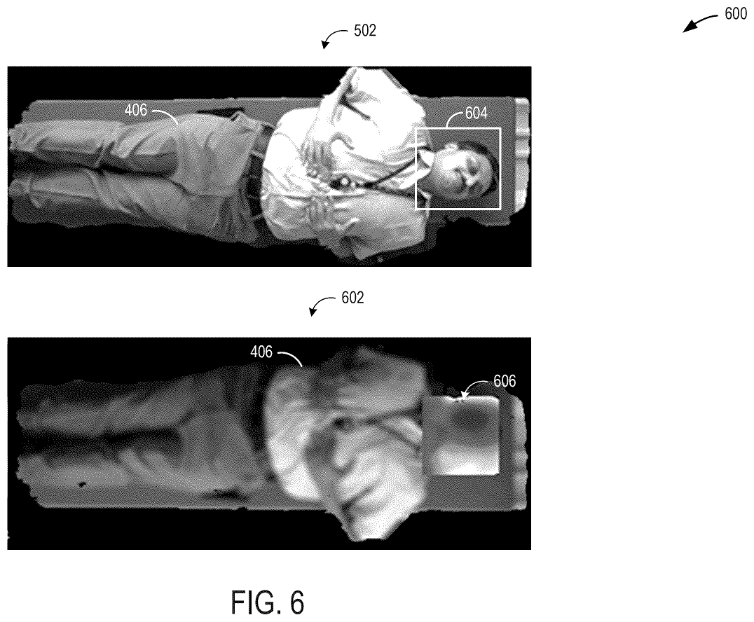

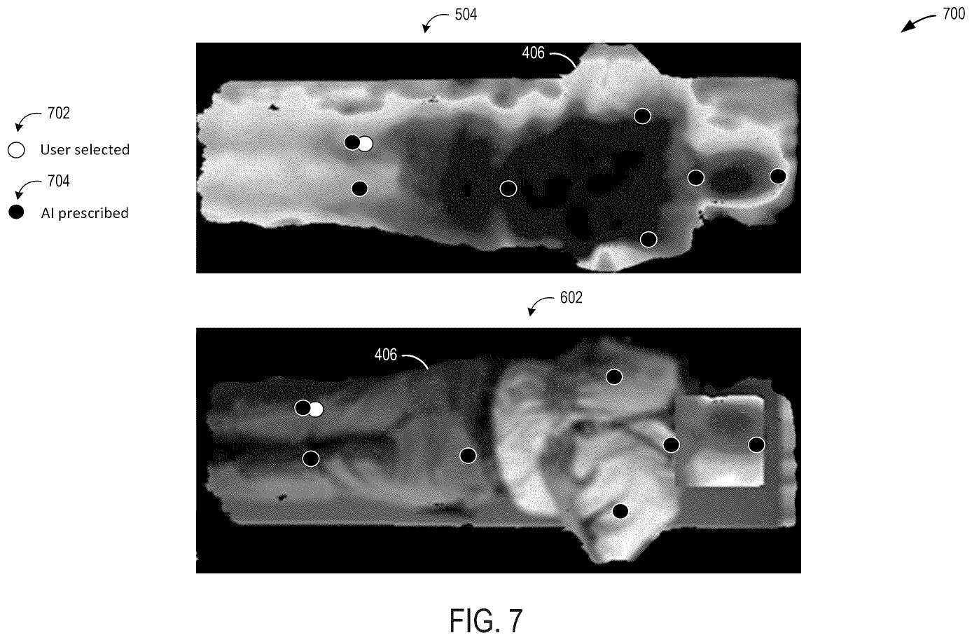

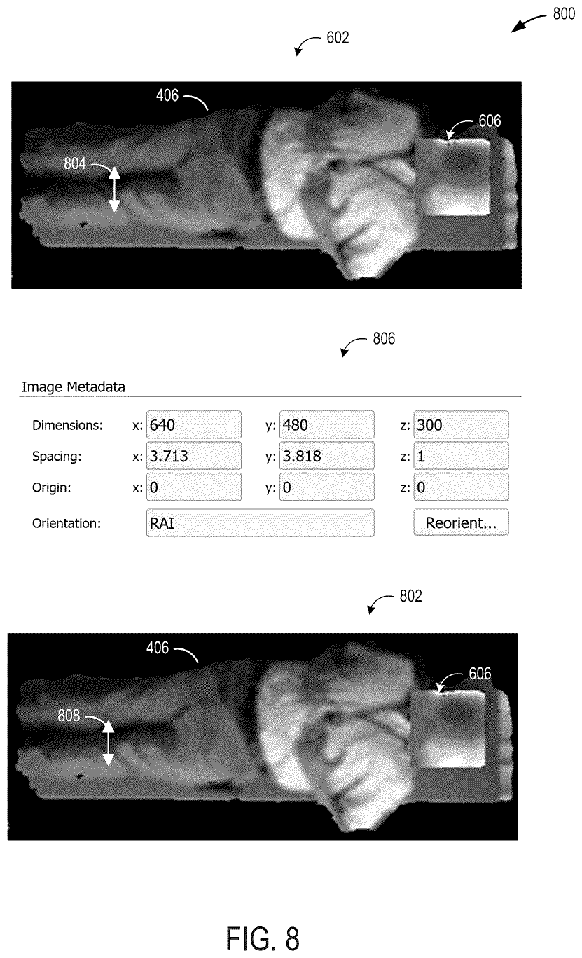

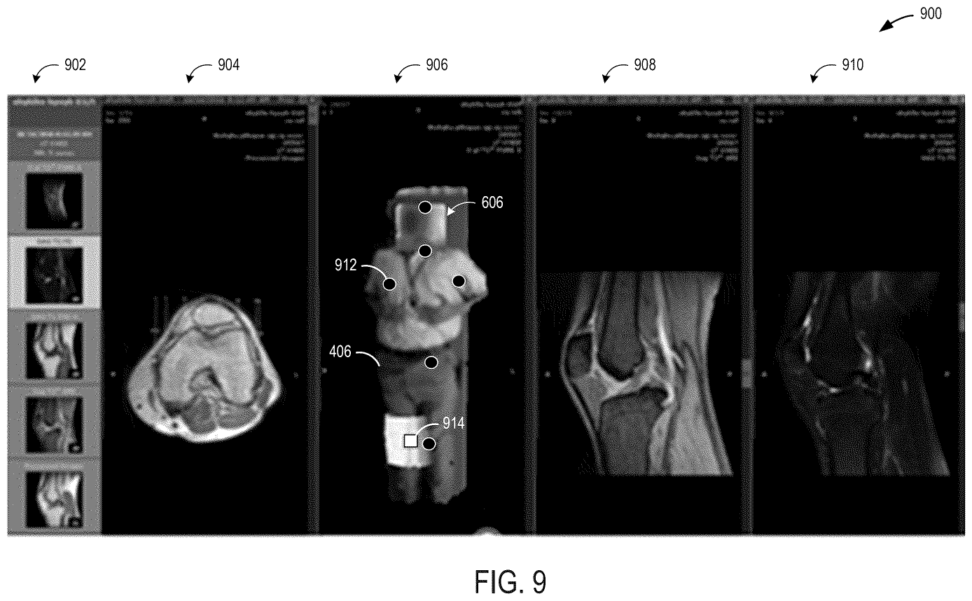

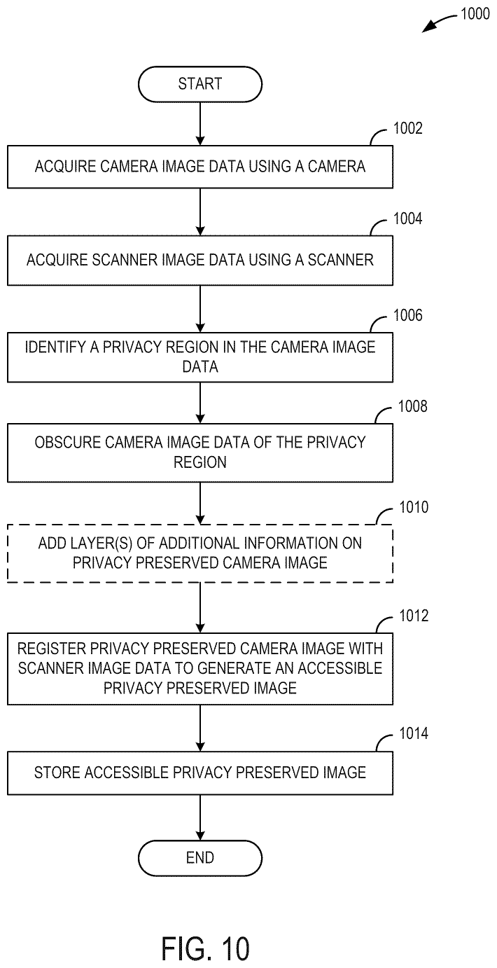

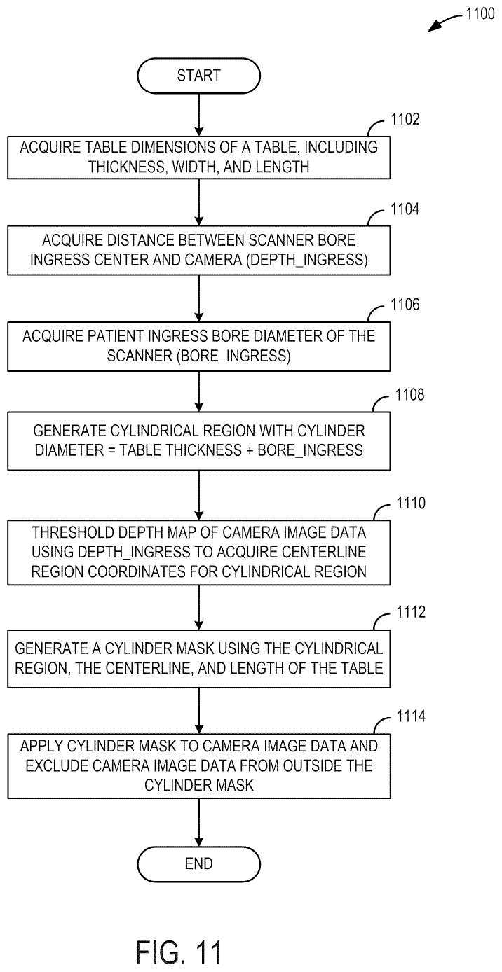

The following description relates to an imaging system configured to preserve patient privacy in captured and stored diagnostic images. In one example, the imaging system is a CT imaging system, an example of which is illustrated in FIGS. 1, 2, and 12. FIG. 3 shows a set of images illustrating conventional image capture, which may not include camera image data or other information about patient positioning. FIG. 4 shows an example setup of a camera configured for camera image data capture and an example camera image captured by the camera. FIG. 5 shows example images of RGB image data and depth map image data included in the camera image data captured by the camera. FIG. 6 shows example placement of a privacy region bounding box on RGB image data, and a privacy preserved camera image with camera image data obscured in the privacy region. FIG. 7 shows example placement of user-selected and AI-prescribed landmarks on depth map image data and the privacy preserved camera image. FIG. 8 shows example images of the privacy preserved camera image and an accessible privacy preserved image. FIG. 9 shows a set of images illustrating image capture for an accessible privacy preserved image. FIG. 10 shows a flowchart of a method for generating an accessible privacy preserved image that includes information captured by a camera and by a diagnostic scanner. FIG. 11 shows a flowchart of a method for a table retention algorithm that may be used in the method of FIG. 10 to identify a privacy region in the camera image data.

Described herein is a method for privacy enabled capture of a camera-based patient setup that includes information captured by both a camera and a diagnostic image scanner. Information includes imaging subject position, landmarks detected, final landmarks selected by a user, electrode placement, MR coil placement, and so on. Personally identifying information of the camera image is obscured in a privacy region to preserve privacy of the imaging subject when storing the image. The method further enables navigation of the image using real-world coordinates in a standard medical imaging format (e.g., DICOM®) viewer (e.g., where object scale/size is true to real world size). The method enables capture and storage of a camera-based setup while protecting patient privacy by obscuring private regions. The camera-based setup may be retrieved from storage and reviewed to determine a quality of camera-based placement/patient setup. This enables the user and/or reviewer to quickly visualize a snapshot of the camera-based setup.

The systems and methods described herein enable retrospective viewing (e.g., post imaging procedure/image capture) of the camera-based patient setup used to scan an imaging subject (e.g., a patient). The retrospective viewing provides data privacy preservation for patients and hospital administration. Further, the systems and methods provide assisted interpretation of user behavior for use in a final setup (e.g., over-riding default provided by artificial intelligence (AI) algorithms). Additionally, the systems and methods enable repeated use of the camera-based patient setup for future imaging procedures. The systems and methods described herein may be particularly useful in training and assisting new technologists who are unfamiliar with patient positioning demands of various imaging procedures. For example, the systems and methods described herein may be used in imaging procedures that employ endorectal coils in prostate due to brightness form the coil, and spinal cord visibility planning. Privacy concerns encountered when storing images may be overcome by image blending or depth-based data capture of a camera-based patient setup. These methods are provided within DICOM® framework as an example of a standard medical imaging format. It is to be understood that the methods may be applied within other standard medical imaging formats with the ability to perform real-world navigation and enable image metrology, without departing from the scope of the present disclosure. In some examples, the method includes using additional channels of information, such as IR, or registering the image to a mesh atlas and storing as a 3D STL model of the patient.

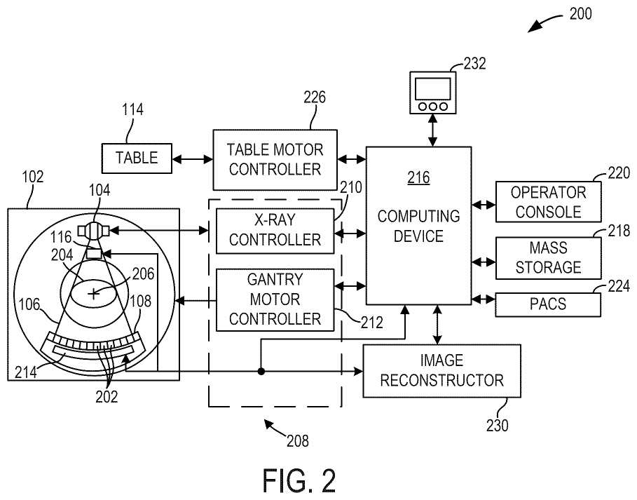

FIG. 1 illustrates an exemplary CT imaging system 100 configured for CT imaging. Particularly, the CT imaging system 100 is configured to image a subject 112 such as a patient, an inanimate object, one or more manufactured parts, and/or foreign objects such as dental implants, stents, and/or contrast agents present within the body. In one embodiment, the CT imaging system 100 includes a gantry 102, which in turn, may further include at least one x-ray source 104 configured to project a beam of x-ray radiation 106 (see FIG. 2) for use in imaging the subject 112 laying on a table 114. The gantry 102 includes a bore 118 into which the table 114, and thus the imaging subject 112, may be inserted during an imaging procedure.

Returning to FIG. 1, the x-ray source 104 is configured to project the x-ray radiation beams 106 towards a detector array 108 positioned on the opposite side of the gantry 102. Collectively, the x-ray source 104 and the detector array 108 are referred to as a scanner 120. Although FIG. 1 depicts only a single x-ray source 104, in certain embodiments, multiple x-ray sources and detectors may be employed to project a plurality of x-ray radiation beams 106 for acquiring projection data at different energy levels corresponding to the patient. In some embodiments, the x-ray source 104 may enable dual-energy gemstone spectral imaging (GSI) by rapid peak kilovoltage (kVp) switching. In some embodiments, the x-ray detector employed is a photon-counting detector which is capable of differentiating x-ray photons of different energies. In other embodiments, two sets of x-ray sources and detectors are used to generate dual-energy projections, with one set at low-kVp and the other at high-kVp. It should thus be appreciated that the methods described herein may be implemented with single energy acquisition techniques as well as dual energy acquisition techniques.

The CT imaging system 100 further includes an image processor unit 110 configured to reconstruct images of a target volume of the subject 112 using an iterative or analytic image reconstruction method. For example, the image processor unit 110 may use an analytic image reconstruction approach such as filtered back projection (FBP) to reconstruct images of a target volume of the patient. As another example, the image processor unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject 112. As described further herein, in some examples the image processor unit 110 may use both an analytic image reconstruction approach such as FBP in addition to an iterative image reconstruction approach.

In some CT imaging system configurations, an x-ray source projects a cone-shaped x-ray radiation beam which is collimated to lie within an X-Y-Z plane of a Cartesian coordinate system and generally referred to as an “imaging plane.” The x-ray radiation beam passes through an object being imaged, such as the patient or subject. The x-ray radiation beam, after being attenuated by the object, impinges upon an array of detector elements. The intensity of the attenuated x-ray radiation beam received at the detector array is dependent upon the attenuation of a radiation beam by the object. Each detector element of the array produces a separate electrical signal that is a measurement of the x-ray beam attenuation at the detector location. The attenuation measurements from all the detector elements are acquired separately to produce a transmission profile.

The CT imaging system 100 further includes a camera 116 configured to capture red, green, blue (RGB) image data and depth map image data, and thus a camera image, of the subject 112 laying on the table 114. For example, the camera 116 may be a three-dimensional (3D) camera. The camera 116 may be included in the gantry 102. For example, the camera 116 may be positioned on the same side of the gantry 102 as the x-ray source 104 such that a field of view of the camera 116 is directed towards a bore 118 of the gantry 102. In further examples, the camera 116 may be positioned outside of the gantry 102. For example, the camera 116 may be positioned alongside the table 114 and oriented such that a field of view of the camera 116 captures at least part of the table 114, and the imaging subject 112 positioned thereon. The camera 116 may be positioned on a stand, mobile cart, suspended from and/or attached to a ceiling of a room in which the CT imaging system 100 is positioned. The camera 116 may be communicably coupled to the image processor unit 110.

The camera 116 may be integrated into the CT imaging system 100 in a non-conventional way. The camera 116 and the scanner 120 may both be communicably coupled, and send imaging data captured by each element (e.g., camera image data captured via the camera 116, scanner image data captured via the scanner 120), to the image processor unit 110. The image processor unit 110 is configured to execute instructions to register the camera image data and the scanner image data.

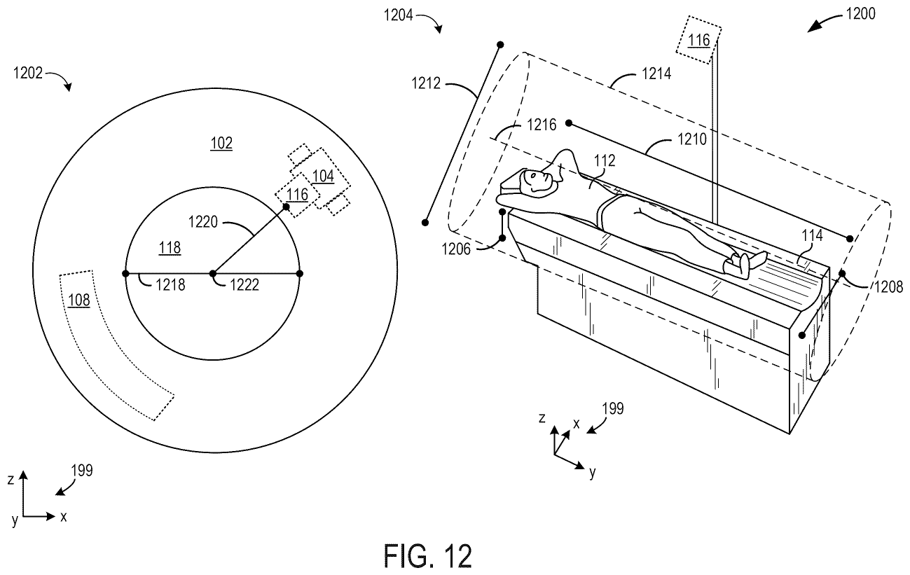

A set of reference axes 199 are provided for comparison between views shown in FIGS. 1 and 12. The reference axes 199 indicate a y-axis, an x-axis, and a z-axis. In one example, the z-axis may be parallel with a direction of gravity. When referencing direction, positive may refer to in the direction of the arrow of the y-axis, x-axis, and z-axis and negative may refer to in the opposite direction of the arrow of the y-axis, x-axis, and z-axis.

Turning briefly to FIG. 12, block diagram schematics 1200 are shown of the exemplary CT imaging system 100 of FIG. 1. A first block diagram 1202 shows the gantry 102 of the CT imaging system 100. The bore 118 has a bore diameter 1218. The gantry 102 further has a first distance 1220 between the camera 116 and a center 1222 of the bore 118. A second block diagram 1204 shows the table 114 of the CT imaging system 100. The table 114 has a thickness 1206, a width 1208, and a length 1210. FIG. 12 further shows a cylindrical region 1214. The cylindrical region 1214 has a cylindrical diameter 1212 that is equal to a sum of the thickness 1206 and the bore diameter 1218. The cylindrical region 1214 further has a centerline 1216. Further details of the cylindrical region 1214 are described with respect to FIG. 11.

The camera 116 may be positioned in the gantry 102, as shown in the first block diagram 1202, and/or outside of the gantry 102, as shown in the second block diagram 1204. A distance between the camera 116 and the table 114 may be adjustable. For example, a height that the camera 116 is positioned (e.g., with respect to the z-axis) may be adjustable to include all or part of the table 114 and/or the imaging subject 112. Further, the camera 116 may be moved closer to or further from the table, with respect to the x-axis). As the table 114 passes through the bore 118 during execution of an imaging procedure, the center 1222 of the bore 118 travels along the centerline 1216. Thus, the first distance 1220 between the camera 116 and the center 1222 of the bore 118 is adjustable.

Returning to FIG. 1, the x-ray source 104, the detector array 108, and the camera 116 may be rotated with the gantry 102 within the imaging plane and around the object to be imaged such that an angle at which the radiation beam intersects the object constantly changes. A group of x-ray radiation attenuation measurements, e.g., projection data, from the detector array at one gantry angle is referred to as a “view.” A “scan” of the object includes a set of views made at different gantry angles, or view angles, during one revolution of the x-ray source and detector. It is contemplated that the benefits of the methods described herein accrue to medical imaging modalities other than CT, so as used herein the term “view” is not limited to the use as described above with respect to projection data from one gantry angle. The term “view” is used to mean one data acquisition whenever there are multiple data acquisitions from different angles, whether from a CT, positron emission tomography (PET), or single-photon emission CT (SPECT) acquisition, and/or any other modality including modalities yet to be developed as well as combinations thereof in fused embodiments.

The projection data is processed to reconstruct an image that corresponds to a two-dimensional slice taken through the object or, in some examples where the projection data includes multiple views or scans, a three-dimensional rendering of the object. One method for reconstructing an image from a set of projection data is referred to in the art as the filtered back projection technique. Transmission and emission tomography reconstruction techniques also include statistical iterative methods such as maximum likelihood expectation maximization (MLEM) and ordered-subsets expectation-reconstruction techniques as well as iterative reconstruction techniques. This process converts the attenuation measurements from a scan into integers called “CT numbers” or “Hounsfield units,” which are used to control the brightness of a corresponding pixel on a display device.

To reduce the total scan time, a “helical” scan may be performed. To perform a “helical” scan, the patient is moved while the data for the prescribed number of slices is acquired. Such a system generates a single helix from a cone beam helical scan. The helix mapped out by the cone beam yields projection data from which images in each prescribed slice may be reconstructed.

As used herein, the phrase “reconstructing an image” is not intended to exclude embodiments of the present disclosure in which data representing an image is generated but a viewable image is not. Therefore, as used herein, the term “image” broadly refers to both viewable images and data representing a viewable image. However, many embodiments generate (or are configured to generate) at least one viewable image.

FIG. 2 illustrates an exemplary imaging system 200 similar to the CT imaging system 100 of FIG. 1. In accordance with aspects of the present disclosure, the imaging system 200 is configured for imaging a subject 204 (e.g., the subject 112 of FIG. 1). In one embodiment, the imaging system 200 includes the detector array 108 (see FIG. 1). The detector array 108 further includes a plurality of detector elements 202 that together sense the x-ray radiation beam 106 (see FIG. 2) that pass through the subject 204 (such as a patient) to acquire corresponding projection data. Accordingly, in one embodiment, the detector array 108 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the x-ray source 104 and the detector array 108 rotate, the detector array 108 collects data of the attenuated x-ray beams. The data collected by the detector array 108 undergoes pre-processing and calibration to condition the data to represent the line integrals of the attenuation coefficients of the scanned subject 204. The processed data are commonly called projections.

In some examples, the individual detectors or detector elements 202 of the detector array 108 may include photon-counting detectors which register the interactions of individual photons into one or more energy bins. It should be appreciated that the methods described herein may also be implemented with energy-integrating detectors.

The acquired sets of projection data may be used for basis material decomposition (BMD). During BMD, the measured projections are converted to a set of material-density projections. The material-density projections may be reconstructed to form a pair or a set of material-density map or image of each respective basis material, such as bone, soft tissue, and/or contrast agent maps. The density maps or images may be, in turn, associated to form a volume rendering of the basis material, for example, bone, soft tissue, and/or contrast agent, in the imaged volume.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In one embodiment, the imaging system 200 includes a control mechanism 208 to control movement of the components such as rotation of the gantry 102 and the operation of the x-ray source 104. In certain embodiments, the control mechanism 208 further includes an x-ray controller 210 configured to provide power and timing signals to the x-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The DAS 214 may be further configured to selectively aggregate analog data from a subset of the detector elements 202 into so-called macro-detectors, as described further herein. The data sampled and digitized by the DAS 214 is transmitted to a computer or computing device 216. In one example, the computing device 216 stores the data in a storage device 218. The storage device 218, for example, may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the x-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates only one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 200 either includes, or is coupled to, a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control the table 114. The table 114 may be a motorized table, where a motor may be actuated to adjust a position of the table 114. For example, the table 114 may be adjusted among multiple configurations to appropriately position the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized x-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely, and may be operatively connected to the imaging system 200 using a wired or wireless network. Particularly, one exemplary embodiment may use computing resources in a “cloud” network cluster for the image reconstructor 230.

In one embodiment, the image reconstructor 230 stores the images reconstructed in the storage device 218. Alternatively, the image reconstructor 230 may transmit the reconstructed images to the computing device 216 for generating useful patient information for diagnosis and evaluation. In certain embodiments, the computing device 216 may transmit the reconstructed images and/or the patient information to a display or display device 232 communicatively coupled to the computing device 216 and/or the image reconstructor 230. In some embodiments, the reconstructed images may be transmitted from the computing device 216 or the image reconstructor 230 to the storage device 218 for short-term or long-term storage.

Though a CT system is described by way of example, it should be understood that the present technology may also be used on other imaging modalities, such as x-ray imaging systems, magnetic resonance imaging (MRI) systems, nuclear medicine imaging systems, positron emission tomography (PET) imaging systems, single-photon emission computed tomography (SPECT) imaging systems, ultrasound imaging systems, and combinations thereof (e.g., multi-modality imaging systems, such as PET/CT or PET/MR imaging systems). The present discussion of a CT imaging modality is provided merely as an example of one suitable imaging modality.

FIG. 3 shows a series of images 300 illustrating example images captured via traditional setups using localizers. A first image 302 is a scout/localizer scan image that may be captured by the scanner (e.g., the CT imaging system 100) prior to a diagnostic imaging scan. A second image 304 and a third image 306 provide screen captures of planned scan prescription for diagnostic imaging series. The second image 304 and the third image 306 may be generated based on the first image 302. However, a scout-based capture, as shown in the third image 306, may not support measurements and may not synchronize with imaging data in a standard medical imaging format (e.g., DICOM®) viewer. A fourth image 308 is an image of a diagnostic imaging series.

Images captured using traditional setups as described with respect to FIG. 3 may not provide information about a position of the imaging subject in real-world space, nor may they provide information about the imaging procedure setup (e.g., placement of coils, electrodes, and so on). Information about imaging procedure setup and patient position may be desirable to capture and store, as it may assist in diagnostic analysis of diagnostic images, as well as recreation of an imaging procedure configuration including imaging system setup and patient position for execution of future imaging procedures. This information may be captured by a camera image. However, the camera image may also capture personally identifying information of an imaging subject (e.g., facial features). For privacy preserving purposes, it may be desirable to not store the personally identifying features. However, for imaging purposes described above, it may be desirable to store the camera image. Described herein are systems and methods that enable generation and storage of an accessible privacy preserved image that includes camera image data, obscures privacy regions in the generated image, and is accessible via a standard medical imaging format. Briefly, the method comprises acquiring camera image data via a camera and acquiring scanner image data via a scanner, identifying a privacy region in the camera image data, obscuring camera image data in the privacy region to generate a privacy preserved camera image, and registering the privacy preserved camera image with scanner image data using a camera-scanner coordinate transformation matrix to generate an accessible privacy preserved image.

FIG. 4 shows a second set of images 400 illustrating an example setup of a camera configured to capture camera image data, and an example camera image captured by the camera. FIG. 4 may be described with respect to FIGS. 1-2. For example, the camera may be an example of the camera 116 of FIGS. 1-2. A schematic 410 illustrates the example setup of the camera 402 with respect to the imaging subject 406 (e.g., the subject 112) positioned on the table 408 (e.g., the table 114). The camera 402 may be directed towards the imaging subject 406 such that a camera field of view 404 captures camera image data of at least part of the imaging subject 406. The camera image data may include RGB image data and depth map image data.

The camera image data captured by the camera 402 may be used to generate a camera image 412. Described another way, the camera 402 may capture the camera image 412. Consent has been obtained from the internal General Electric Healthcare™ volunteer to show his face for demonstration in the present disclosure. The camera image 412 is shown in greyscale for the purposes of the disclosure, though it is to be understood that the camera image 412 may be in color. The camera image 412 includes the imaging subject 406, the table 408, a diagnostic imaging system 416 (e.g., the CT imaging system 100) and elements of a surrounding environment 414 of the table 408 and the imaging subject 406. For example, the surrounding environment 414 may be a medical exam room, and the camera image 412 may include a floor 418, chairs 420, and a camera stand 422 of the camera 402.

FIG. 5 shows a third set of images 500, including the camera image 412 of FIG. 4, an RGB image 502 generated from RGB image data of the camera image 412, and a depth map 504 generated from the depth map image data of the camera image 412. The table retention algorithm is applied to the camera image data to identify a table region of interest that includes the table 408 and the imaging subject 406 positioned on the table 408, as well as parts of the imaging subject 406 that may extend beyond bounds of the table 408 (e.g., beyond a width 506 of the table 408). The table retention algorithm removes camera image data from areas outside of the table region of interest, as further described with respect to FIGS. 10-11. Thus, at least one camera image is generated by the table retention algorithm that includes RGB image data and depth map image data of the imaging subject 406 and the table 408, and excludes RGB image data and depth map image data from other regions in the camera image data. The RGB image 502 and the depth map image 504 are examples of camera images generated by the table retention algorithm that show camera image data of the table region of interest.

FIG. 6 shows a fourth set of images 600, including example placement of a privacy region bounding box 604 on the RGB image 502, and a privacy preserved camera image 602 with camera image data obscured in a privacy region. As is further described with respect to FIGS. 10-11, a landmark detection algorithm may be used to identify and define a privacy region (e.g., a face of the imaging subject 406). The privacy region may be outlined in the camera image data by the privacy region bounding box 604. Camera image data in the privacy region may be obscured, such that personally identifying information of the imaging subject may not be visible. For example, the RGB image data and the depth map image data may be fused in the privacy region bounding box 604 to generate a privacy preserved region 606. Thus, the face of the imaging subject 406 is obscured. In other examples, the privacy region may include other personally identifying features, such as tattoos and/or birthmarks. In further examples, more than one privacy region may be identified, and camera image data may be obscured in each privacy region.

FIG. 7 shows a fifth set of images 700 including example placement of additional information on the depth map image 504 and the privacy preserved camera image 602. The additional information may include one or more user-selected landmarks 702 (white circle with black outline) and/or one or more artificial intelligence (AI)-prescribed landmarks 704 (black circle with white outline). Additional information may be placed on the depth map image 504 and/or the privacy preserved camera image 602 as layers on the data as pixels burnt into the image. For example, adding additional layers of information may comprise embedding burnt-in pixels into the privacy preserved camera image. In the example of FIG. 7, the landmarks may illustrate anatomy of the imaging subject 406. For example, a landmark detection algorithm may be an AI algorithm configured to identify and label regions of the body. The AI algorithm may assign a landmark at each of a left knee, a right knee, a left hip, a right arm, a left arm, a chin, and a top of a head of the imaging subject 406. Additionally or alternatively, the landmarks may illustrate a position of a coil placement, electrode placement, and so on.

A user (e.g., an imaging technologist, a medical provider) may add one or more landmarks to the camera images. For example, the user may adjust a position of a landmark for the right knee, which may be an imaging area of interest. The landmark detection algorithm may be further configured to compare the user-selected landmark and the AI-prescribed landmark for the same region, and output a potential reasoning for the difference between the user-selected and AI-prescribed landmark positions.

FIG. 8 shows a sixth set of images 800 including the privacy preserved camera image 602 and an accessible privacy preserved image 802. In the example described herein with respect to FIGS. 3-9, the accessible privacy preserved image 802 is a DICOM®-accessible privacy preserved image. The accessible privacy preserved image 802 may thus be configured to be viewed and interacted with (e.g., measured, annotated, etc.) via the DICOM® framework, a DICOM® viewer, and so on. As further described with respect to FIGS. 10-11, the accessible privacy preserved image 802 may be compatible with one or more additional or alternative standard medical imaging formats.

The privacy preserved camera image 602 is generated from camera image data captured by the camera, including RGB image data and depth map image data. Camera image data in the privacy preserved region 606 is obscured to obfuscate personally identifying information of the imaging subject 406. The privacy preserved camera image 602 is shown in greyscale for the purposes of the disclosure, though it is to be understood that the privacy preserved camera image 602 may be in color. The privacy preserved camera image 602 may be a portable network graphic (PNG) image that includes pixel information. Thus, the privacy preserved camera image 602 may be navigated using a pixel coordinate system. For example, a distance 804 between a left knee and a right knee of the imaging subject 406 as measured in the privacy preserved camera image 602 may be 52 pixels. Scanner image data captured by a diagnostic scanner (e.g., the CT system/x-ray source of FIGS. 1-2) may use a real-world coordinate system with dimensions of a scanner image measured as a physical distance, such as in millimeters.

The privacy preserved camera image 602 may be registered with scanner image data to generate the accessible privacy preserved image 802. Registering camera image data and scanner image data, which have two different coordinate systems, may include generating a geometry header 806 using a camera-scanner coordinate system matching. The camera image data (e.g., the privacy preserved camera image 602) may be transformed to an accessible save state (e.g., such that camera image data is accessible via the standard medical imaging format). A coordinate transformation matrix (e.g., a camera-scanner calibration coordinate system) may be established to link the privacy preserved camera image and a scanner image comprising scanner image data captured by the scanner, generating the accessible privacy preserved image 802. Thus, real-world physical measurements may be performed on camera image data, via the accessible privacy preserved image 802. For example, a distance 808 between the left knee and the right knee of the imaging subject 406 may be measured as 190 mm, which has a more real-world application than the pixel distance of 52 pixels. Further, the accessible privacy preserved image 802 may be synchronized with other scanner image data and scanner images that are accessible via the standard medical imaging format, such as DICOM®. Generation of an accessible privacy preserved image is further described with respect to FIG. 10-11.

FIG. 9 shows an example view 900 illustrating storage of camera workflow information as a DICOM®-accessible privacy preserved image. The example view 900 may be an example of a DICOM® viewer displayed on a display device, such as a desktop computer, tablet, mobile device, or other display device that is communicably coupled to the imaging system and to the computing device executing the method for generating and storing the DICOM®-accessible privacy preserved image. A first panel 902 shows an overview of captured and/or generated images. A second panel 904 shows a scout capture. For example, the scout capture may be an image captured by the scanner prior to execution of a diagnostic scan. A third panel 906 shows a DICOM®-accessible privacy preserved image that may be generated using the methods described herein with respect to FIGS. 4-8 and further described with respect to FIGS. 10-11. The DICOM®-accessible privacy preserved image of FIG. 9 has camera image data obfuscated in the face area of the imaging subject, and further includes AI-prescribed landmarks 912 and a user-selected region of interest 914. A fourth panel 908 shows a first DICOM® view (e.g., diagnostic scan image) of the user-selected region of interest 914, and a fifth panel 910 shows a second DICOM® view of the user-selected region of interest 914.

The systems and methods described herein provide technical solutions to challenges in imaging system technology and specifically solutions directed to camera and diagnostic scanner image processing. In particular, a coordinate transformation matrix is used to register camera image data and scanner image data following processing of the camera image data. Registration using the coordinate transformation matrix involves specialized mathematical operations that go beyond generic computer implementation. The camera-scanner coordinate transformation matrix is generated through a calibration process that accounts for physical positioning parameters of the imaging subject with respect to the scanner, via the camera. For example, the camera-scanner coordinate transformation matrix provides: camera focal length and optical parameters; physical distances between the camera, scanner bore, and table; geometric distortion correction factors; and scanner-specific spatial reference points. In this way, camera image data and scanner image data may be registered in such a way that preserves information of each image data in a single, accessible privacy preserved image. Registration of the camera image data and the scanner image data goes beyond generic computer implementation, as one or both of the camera image data and the scanner image data may be processed and/or transformed as described herein to be compatible for registration with the other image data.

FIG. 10 shows a flow chart for a method 1000 for generating an accessible privacy preserved image that includes information captured by a camera and by a diagnostic scanner. The method 1000 may be executed by a computing device that is communicably coupled to an imaging system comprising a camera and a diagnostic scanner, and/or a database that stores image data captured by an imaging system comprising a camera and a diagnostic scanner. For example, the method 1000 may be executed by the CT imaging system 100 of FIGS. 1-2. The method 1000 may be stored in a computing device (e.g., the computing device 216) having instructions stored on non-transitory memory and executable by a processor.

At 1002, the method 1000 comprises acquiring camera image data captured by a camera. In examples where the method 1000 is executed by an imaging system comprising a camera and a diagnostic scanner, acquiring camera image data may include capturing a camera image comprising camera image data, using the camera. In examples where the method 1000 is executed by a computing device that is communicably coupled to a database storing image data captured by the imaging system, acquiring camera image data may include sending a query to the database requesting camera image data, and receiving camera image data from the database. Camera image data may include depth information and red, green, blue (RGB) image data. Camera image data provides information about a camera-based setup of an imaging scan. For example, in different imaging setups, such as to image different regions of the imaging subject, one or more of the camera, the imaging subject, imaging system coils, and/or electrodes may have different positions. Referring briefly to FIG. 4, the imaging subject 406 is positioned on their back with their arms on their torso and legs flat. The camera is positioned on a left side of the imaging subject and the field of view of the camera captures all of the body of the imaging subject except for a small portion of the feet. In other imaging setups, such as to capture diagnostic scans of the torso, arms of the imaging subject may be extended above their head and the camera may be positioned to have the field of view exclude areas of the imaging subject below the waist and focus instead on detailed positioning of the torso of the imaging subject. In further examples of imaging setups, one or more of the legs of the imaging subject may be bent, electrodes may be positioned on one or more of the legs, and the camera may be positioned to capture a location of one or more electrodes.

At 1004, the method 1000 comprises acquiring scanner image data captured by a diagnostic scanner. In examples where the method 1000 is executed by an imaging system comprising a camera and a diagnostic scanner, acquiring scanner image data may include capturing a scanner image comprising scanner image data, using the diagnostic scanner. In examples where the method 1000 is executed by a computing device that is communicably coupled to a database storing image data captured by the imaging system, acquiring scanner image data may include sending a query to the database requesting scanner image data, and receiving scanner image data from the database. Scanner image data may provide a diagnostic image that is configured in a standard medical imaging format, such as DICOM®.

At 1006, the method 1000 comprises identifying a privacy region in the camera image data. The privacy region is a region that may include personally identifying information of an imaging subject. For example, the privacy region may include a face of the imaging subject. To preserve privacy and anonymity of the imaging subject, it may be desirable to obscure camera image data within the privacy region such that identifying features of the imaging subject may not be discernable. Identifying the privacy region may include executing a table retention algorithm to localize camera image data to within a table region, where the table region includes a table of the imaging system on which the imaging subject is positioned. The table retention algorithm may be configured to remove camera image data from outside of the table region. Identifying the privacy region may further include applying a landmark detection algorithm.

The landmark detection algorithm may be any one or more of an algorithm configured to identify landmarks on RBG images and/or depth images (e.g., as included in the camera image data). For example, the landmark detection algorithm may be an open source artificial intelligence (AI)-based model for human landmark and pose detection that may also be used for anatomy landmark detection. The landmark detection algorithm may be applied to RGB image data of the camera image data to identify anatomy landmarks that are used as bounds of the privacy region. For example, a top of a head and a neck of the imaging subject may each be anatomy landmarks with relatively known positions that are identified as superior-inferior bounds of the privacy region. Additional anatomy landmarks may be cars of the imaging subject (e.g., car landmarks), and may be identified and used as lateral bounds of the privacy region. A bounding box may be generated using the identified bounds. The bounding box may be used to crop depth map information of the camera image data to generate the privacy region. As the RGB image data and the depth map information are captured by the camera, the privacy region is mapped onto the RGB image data by aligning the depth map and the RGB image data without demand for further processing. Each pixel of the camera image data (e.g., including RGB image data and depth map image data for the respective pixel) may be assigned a label that indicates whether or not the pixel is in the bounding box, and therefore in the privacy region.

At 1008, the method 1000 includes obscuring camera image data of the privacy region. Obscuring camera image data may remove identifiable features of the imaging subject from the camera image data, and therefore from an accessible image generated by the method 1000. Camera image data of the privacy region may be obscured by fusing RGB image data and depth map image data. For example, a face region from the depth map image data within the bounding box may be blended with the RGB image data. Camera image data may additionally or alternatively be obscured by blending camera image data. For example, one or more of alpha blending, Poisson blending, or AI-based blending may be used to obscure camera image data of the privacy region. Poisson blending and AI-based blending may be performed according to conventional methods.

Alpha blending is an example of a non-AI-based blending method, and may include fusing a foreground image (e.g., depth map image data of the privacy region) and a background image (e.g., RGB image data of the privacy region). For example, fusion of the foreground image and the background image may be controlled using a first equation (1),

$$I = α F + ( 1 - α ) B \qquad ( 1 )$$

where I is a fused image, F is the foreground image, B is the background image, and α is a blending factor. The blending factor may have different values for different regions within the camera image data. For example, within the privacy region (e.g., the face region), the blending factor may be α=1.0, and outside of the privacy region, the blending factor may be α=0. The first equation may be applied to each pixel of the camera image data using a corresponding blending factor. For example, a first pixel of the camera image data may be determined to be in the privacy region (e.g., as described with respect to operation 1006), and the first equation may be applied to camera image data (e.g., RGB image data and depth map image data) of the first pixel, using the blending factor that corresponds to the privacy region (e.g., α=1.0). A second pixel of the camera image data may be determined to be outside of the privacy region (e.g., a part of the imaging subject that is not the face of the imaging subject), and the first equation may be applied to camera image data of the second pixel using the blending factor that corresponds to regions that are not the privacy regions. In this way, the entirety of the camera image data may be quickly processed by assigning a corresponding blending factor to each pixel within the camera image data, and applying the first equation to all pixels of the camera image data. Alternatively, the first equation may be applied to camera image data of pixels in the privacy region and may not be applied to camera image data of pixels that are outside of the privacy region. This may decrease a processing time, a processing demand, a memory demand, and amount of data to be stored.

In some examples, camera image data of the privacy region may be obscured by applying a privacy preserving depth channel. This may include storing and/or otherwise retaining the depth map of pixels within the privacy region of the camera image data, and not storing and/or otherwise retaining the RGB image data of pixels within the privacy region of the camera image data. Since depth map image data may not contain facial features or otherwise personally identifying information, not including the RGB image data in stored camera image data for the privacy region may sufficiently obscure identifiable features of the imaging subject. Compared to obscuring camera image data by blending, as described above, excluding RGB image data from the privacy region to obscure camera image data may decrease a processing time and processing demand, as well as decrease memory and data storage demand.

In further examples, camera image data of the privacy region may be obscured by registering the imaging subject to a digital body atlas using landmarks (e.g., identified using the landmark detection algorithm described above) and the privacy region (e.g., the bounding box of the privacy region), and obscuring the privacy region in the digital body atlas. The digital body atlas may include pre-defined anatomical landmarks. The bounding box of the camera image data, including the RGB image data, may be scaled and mapped to the digital body atlas. For example, the RGB image data may be matched in scale to the digital body atlas using an Affine transformation. The privacy region bounding box of the digital body atlas may be pasted to the scaled RGB image data using the blending procedure described above (e.g., the first equation).

In some examples, the depth map image data may not be registered to the digital body atlas. As the depth map image data may not include personally identifying information, the RGB image data of the camera image data can be used with the digital body atlas to obscure the privacy region. This may reduce a processing demand and processing time, compared to methods that map both depth map and RGB image data with the digital body atlas.

In this way, a camera image with an obscured privacy region (herein, “the privacy preserved camera image”) may be generated from the camera image data (e.g., the RGB image data and the depth map image data), where features of the imaging subject that appear in the privacy preserved camera image and may be personally identifiable are obscured. Features captured by the camera and that may not be captured by the scanner, such as a relative positioning of the imaging subject in the imaging system, are thus retained while excluding features of the imaging subject that may be private. Privacy region detection and obscuring is thus performed using a specialized algorithm that includes multi-scale feature detection to identify anatomical landmarks, geometric relationship analysis between detected features, machine learning models trained on anatomical feature datasets, and dynamic adjustment of detection parameters based on imaging conditions. Novel technical approaches are further implemented to obscure data within the privacy region. The method 1000 includes selective depth-map retention while removing RGB data, multi-resolution blending with spatially-varying blend factors, and real-time verification of privacy preservation effectiveness.

At 1010, the method 1000 optionally includes adding one or more layers of additional information to the privacy preserved camera image. The additional information may include AI and/or user-selected landmarks, magnetic resonance (MR) coil setup of the imaging system, respiratory bellows, electrode setup, and/or other details that may assist a user (e.g., a medical technician and/or practitioner) in interpreting imaging data and/or recreating imaging conditions (e.g., relative positioning of the imaging subject, coil placement) during future imaging procedures. Adding one or more layers of additional information to the privacy preserved camera image may include pasting each layer onto the camera image using conventional methods for applying a mask to an image.

At 1012, the method 1000 includes registering the privacy preserved camera image with scanner image data. Camera-scanner coordinate system matching may be performed to transform the camera image to an accessible save state (e.g., accessible via one or more standard medical imaging formats. Calibration of the privacy preserved camera and the scanner (e.g., generation of the camera-scanner coordinate system) may be performed prior to execution of the method 1010. A coordinate transformation matrix (e.g., a camera-scanner calibration coordinate system) may be established to link the privacy preserved camera image and a scanner image comprising scanner image data captured by the scanner.

Calibration of the privacy preserved camera image captured by the camera, and the scanner image (e.g., a diagnostic image) captured by the scanner enables linking of RGB image data and diagnostic imaging data using physical coordinates. Standard camera calibration routines may map pixel distances to physical measurements. For example, a physical distance between the table and the camera may be established, and one or more fiducial markers may be placed on the table at known locations. Distances of each fiducial marker with respect to each other and to the camera in both a vertical and a horizontal plane are known. Pixel dimensions with respect to physical distance (e.g., in millimeters (mm) may be computed as a distance between any two fiducials using a second equation (2),

$$\frac{p i x}{mm} ratio = \frac{distance in pix}{distance in mm} . \qquad ( 2 )$$

The pixel dimensions

$$( \frac{p i x}{mm} ratio )$$

may be applied to a pixel measurement (dₚᵢₓ) in an image domain to find a physical distance in millimeters. For example, the privacy preserved camera image may be scaled in dimension of pixels, and the scanner image data may be scaled in dimensions of millimeters. The physical distances (dₘₘ) in the privacy preserved camera image may be found using a third equation (3),

$${d}_{m m} = {d}_{pix} / ( \frac{pix}{mm} ratio ) . \qquad ( 3 )$$

In this way, a camera-scanner coordinate system is generated that may be applied to camera image data of the privacy preserved camera image to convert the privacy preserved camera image to physical distances and provide a real-world geometry of the imaging subject in the privacy preserved camera image. A standard medical imaging format-compliant geometry header may be generated using the camera-scanner coordinate system. The geometry header may include, for any pixel of the privacy preserved camera image, image origin coordinates, image direction cosines, and image spacing in millimeters. The geometry header thus enables the privacy preserved camera image to be synchronized with standard-acquired images (e.g., the scanner image). Camera pixel coordinates may be converted to physical, real-world coordinates using calibration parameters and the camera-scanner coordinate system. For example, a physical coordinate is generated by applying a focal length factor and a distance factor to the pixel coordinate, and further applying a distortion correction. The camera-scanner coordinate system may be applied as a transformation matrix that incorporates rotation, translation, and scaling parameters determined during system calibration. A validation of registration accuracy may be performed in real-time using the fiducial markers, which have known physical positions.

The privacy preserved camera image may be linked with the scanner image using physical coordinates of the camera image (e.g., transformed from pixel coordinates using the third equation) and physical coordinates of the scanner image (e.g., intrinsic to the scanner). By virtue of the calibration step, the physical coordinates of each of the scanner image and the camera image use the same coordinate system. Effectively, each voxel in the scanner image and the camera image have the same physical coordinate value. In this way, combining the camera image (with the obscured privacy region) and the scanner image generates an accessible privacy preserved image. For example, the accessible privacy preserved image may be a DICOM®-accessible, privacy preserved image.

At 1014, the method 1000 includes storing the accessible privacy preserved image that includes camera image data, obscures privacy regions in the generated image, and is accessible via a standard medical imaging format. The accessible privacy preserved image may further include additional imaging information, such as coil placement and other landmarks. A standard medical imaging format (e.g., DICOM®) viewer may be used to access and navigate the accessible privacy preserved image using real-world coordinates to enable measurements between anatomical areas of interest, landmarks, and so on. Further, the accessible privacy preserved image may be stored in a standard medical imaging format database. The method 1000 ends. The method 1000 thus transforms raw image data into a new format that enables novel technical capabilities that are not previously possible and that go beyond mere data manipulation.

FIG. 11 shows a flow chart for a method 1100 for a table retention algorithm configured to localize camera image data to within a table region and remove camera image data from outside of the table region from a camera image. The table retention algorithm may be used in part to identify a privacy region in the camera image. For example, the method 1100 may illustrate steps for executing the table retention algorithm of operation 1006 of the method 1000. The method 1100 may be executed by the CT imaging system 100 of FIGS. 1-2. The method 1100 may be stored in a computing device (e.g., the computing device 216) having instructions stored on non-transitory memory and executable by a processor. The method 1100 may be performed when the table is docked in the imaging system in a ready-to-scan position (e.g., the table 408 of the CT imaging system 100 of FIG. 1).

At 1102, the method 1100 includes acquiring table dimensions of the table, including a thickness, a width, and a length of the table. For example, the table dimensions include the thickness 1206, the width 1208, and the length 1210 of the table 114 of FIG. 12. Table dimensions may be system specific, and thus may be known and stored in a memory (e.g., memory of the CT imaging system 100 of FIG. 1) as part of a system calibration file. When the method 1100 is executed by a computing device of the imaging system, operation 1102 may include accessing the memory of the imaging system to retrieve table dimension data. When the method 1100 is executed by a computing device that is communicably coupled to the imaging system, operation 1102 may include sending a request to the imaging system to retrieve the table dimensions from the memory of the imaging system, and receiving the table dimension data.

At 1104, the method 1100 includes acquiring a distance between a scanner bore ingress center and the camera, herein referred to as “depth_ingress”. The depth_ingress may be an example of the first distance 1220 between the camera 116 and the center of the scanner bore 1222 ingress of FIG. 12. Similar to the table dimensions, the depth_ingress may be automatically computed by the computing device of the imaging system during setup and calibration of the imaging system, and thus may be known and stored in a memory as part of the system calibration file. When the method 1100 is executed by a computing device of the imaging system, operation 1104 may include accessing the memory of the imaging system to retrieve the depth_ingress value. When the method 1100 is executed by a computing device that is communicably coupled to the imaging system, operation 1104 may include sending a request to the imaging system to retrieve the depth_ingress value from the memory of the imaging system, and receiving the depth_ingress value.

At 1106, the method 1100 include acquiring a patient ingress bore diameter of the scanner, herein referred to as “bore_ingress”. The bore_ingress may be an example of the bore diameter 1218 of the bore 118 of FIG. 12. Similar to the table dimensions and the depth_ingress, the bore_ingress may be system specific, and thus may be known and stored in a memory as part of the system calibration file. When the method 1100 is executed by a computing device of the imaging system, operation 1106 may include accessing the memory of the imaging system to retrieve the bore_ingress value. When the method 1100 is executed by a computing device that is communicably coupled to the imaging system, operation 1106 may include sending a request to the imaging system to retrieve the bore_ingress value from the memory of the imaging system, and receiving the bore_ingress value.

At 1108, the method 1100 includes generating a cylindrical region with a cylindrical diameter that is equal to a sum of the table thickness and the bore_ingress. The cylindrical diameter of the cylindrical region is thus larger than the width of the table, which enables a table region defined by the table retention algorithm to include parts of an imaging subject that may extend beyond bounds of the table (e.g., arms and/or elbows of the imaging subject, as described with respect to FIG. 5).

At 1110, the method 1100 includes applying a threshold to the depth map image data of the camera image data using the depth_ingress to acquire centerline region coordinates for the cylindrical region. The depth_ingress is the distance between the scanner bore ingress and the camera, thus applying the depth_ingress as a threshold of the depth map image data may localize depths of the cylindrical region to depths illustrated by the depth map image data. Described another way, the cylindrical diameter of the cylindrical region may be adjusted from a single diameter that is equal to the sum of the table thickness and the bore_ingress to multiple diameters that are equal to differences between the depth_ingress and distances that anatomy of the imaging subject extends towards the camera. A centerline of the cylindrical region may be established as a center of the cylindrical diameter, following adjustment.

At 1112, the method 1100 includes generating a cylinder mask using the cylindrical region, the centerline, and the length of the table. A diameter of the cylinder mask is equal to the diameter of the cylindrical region, and a main axis length of the cylinder mask is equal to the length of the table and parallel to the centerline. The cylinder mask may thus encompass an entirety of the table and an entirety of the imaging subject positioned on the table.

At 1114, the method 1100 includes applying the cylinder mask to the camera image data, and excluding camera image data from outside of the cylinder mask. The table and the imaging subject positioned on the table, as well as parts of the imaging subject that may extend beyond bounds of the table (e.g., beyond the width of the table) may be defined as a table region of interest. Applying the cylinder mask removes camera image data from areas outside of the table region of interest, such that a camera image is generated by the table retention algorithm that includes RGB image data and depth map image data of the imaging subject and the table, and excludes RGB image data and depth map image data from other regions in the camera image data. The landmark detection algorithm described with respect to operation 1006 of FIG. 10 may be applied to the camera image generated by the table retention algorithm.

In this way, a privacy preserved standard medical imaging format-accessible image may be generated and stored. The accessible privacy preserved image includes one or more privacy regions in which camera image data is obscured. Landmarks, coil and electrode placement, and/or other additional information may be burned into, and thus stored with the accessible privacy preserved image (e.g., as burnt-in pixels embedded into the privacy preserved camera image). Technical effects of the methods and systems described herein include a decreased processing time, a decreased processing demand, a decreased memory demand, and/or a decreased amount of data to be stored. The memory demand may be decreased, as the methods described herein generate and output for storage of a single image that includes camera image data of interest, landmarks, and other additional data of interest, while excluding image data from outside of the region of interest. Landmark and other positioning data is burned into the image, thus decreasing a number of files to be stored and/or retrieved for a given imaging procedure. Further, imaging subject privacy is preserved while decreasing the amount of data to be stored. Camera image data in the privacy region is fused or otherwise obscured in such a way that detailed pixel information of the privacy region(s) is not included in the generated accessible privacy preserved image. Processing time and demand for further imaging procedures may be decreased, as the generated accessible privacy preserved image, which is stored in the memory of the imaging system and/or a computing device communicably coupled thereto, may be retrieved as a reference for patient positioning and other imaging procedure setup. This may further reduce a demand for additional imaging scans as a result of inaccurate patient positioning, electrode placement, coil placement, and so on. The systems and methods described herein may enable increased accuracy of patient positioning and enhanced privacy protection while maintaining clinical utility. An accuracy of diagnostic image capture may be increased.

The disclosure also provides support for a method for a medical imaging system, comprising: acquiring camera image data, including red, green, blue (RGB) image data and depth map image data, captured via a camera, acquiring scanner image data captured via a scanner, identifying a privacy region in the camera image data, obscuring camera image data in the privacy region to generate a privacy preserved camera image, registering the privacy preserved camera image with scanner image data, using a camera-scanner coordinate transformation matrix, to generate an accessible privacy preserved image that includes camera image data, scanner image data, obscured camera image data in the privacy region, and is accessible via a standard medical imaging format, and storing the accessible privacy preserved image that can be viewed and navigated within a standard medical imaging format viewer. In a first example of the method, the privacy region in the camera image data is identified using a landmark detection algorithm. In a second example of the method, optionally including the first example, identifying the privacy region in the camera image data using the landmark detection algorithm includes identifying landmarks in RGB image data and/or depth map image data based on anatomy landmarks, and generating a bounding box using the anatomy landmarks. In a third example of the method, optionally including one or both of the first and second examples, the method further comprises: using the bounding box to crop depth map information of the camera image data to generate the privacy region. In a fourth example of the method, optionally including one or more or each of the first through third examples, the privacy region includes a face of a subject, and landmarks used by the landmark detection algorithm to identify the face includes car landmarks to identify left and right bounds, and head to neck landmarks to identify superior and inferior bounds. In a fifth example of the method, optionally including one or more or each of the first through fourth examples, identifying the privacy region comprises applying a table retention algorithm to the camera image data to remove camera image data from outside of a table region. In a sixth example of the method, optionally including one or more or each of the first through fifth examples, applying the table retention algorithm comprises: acquiring table dimensions of a table, including a thickness, a width, and a length of the table, acquiring a depth ingress equal to a distance between a scanner bore ingress center of the scanner and the camera, acquiring a bore ingress equal to a patient ingress bore diameter of the scanner, generating a cylindrical region with a diameter that is equal to a sum of the thickness of the table and the bore ingress, acquiring centerline region coordinates for the cylindrical region by applying the depth ingress to the depth map image data of the camera image data, generating a cylinder mask having the diameter of the cylindrical region, the centerline region coordinates, and a main axis length that is equal to the length of the table, and applying the cylinder mask to the RGB image data and depth map image data of camera image data to remove camera image data other than image data of the table and an imaging subject positioned on the table from the camera image data. In a seventh example of the method, optionally including one or more or each of the first through sixth examples, camera image data in the privacy region is obscured via one or more of alpha blending, Poisson blending, or artificial intelligence (AI)-based blending. In an eighth example of the method, optionally including one or more or each of the first through seventh examples, obscuring camera image data in the privacy region comprises blending RGB image data and depth map image data of the camera image data to remove identifiable features of an imaging subject in the privacy region. In a ninth example of the method, optionally including one or more or each of the first through eighth examples, obscuring camera image data in the privacy region comprises blending depth map image data of the camera image data to remove identifiable features of an imaging subject in the privacy region. In a tenth example of the method, optionally including one or more or each of the first through ninth examples, the standard medical imaging format is Digital Imaging and Communication in Medicine (DICOM®). In an eleventh example of the method, optionally including one or more or each of the first through tenth examples, adding additional layers of information comprises embedding burnt-in pixels into the privacy preserved camera image, the burnt-in pixels identifying user selected and/or AI-prescribed landmarks

The disclosure also provides support for a method, comprising: capturing camera image data, including RGB image data and depth map image data, via a camera, capturing scanner image data via a scanner, identifying a privacy region in the camera image data via a landmark detection algorithm, applying a table-retention algorithm to the camera image data to remove camera image data from outside of a table region, obscuring camera image data in the privacy region to generate a privacy preserved camera image, adding additional layers of information from a scanning procedure to the privacy preserved camera image, registering the privacy preserved camera image with scanner image data, using a camera-scanner coordinate transformation matrix, to generate an accessible privacy preserved image that includes camera image data, scanner image data, obscured camera image data in the privacy region, and is accessible via a standard medical imaging format, and storing privacy preserved camera-based patient setup information as the accessible privacy preserved image that can be viewed and navigated within a standard medical imaging format viewer. In a first example of the method, obscuring the camera image data in the privacy region comprises fusing a foreground image and a background image using a first blending factor for the foreground image and a second blending factor for the background image, where the second blending factor is different from the first blending factor. In a second example of the method, optionally including the first example, the foreground image is the depth map image data and the background image is the RGB image data. In a third example of the method, optionally including one or both of the first and second examples, fusing image data comprises: using a map of facial anatomy to identify a bounding box for a face region, matching in scale the bounding box of the face region and a digital body atlas, pasting a transformed bounding box to the RGB image data using the first blending factor and the second blending factor. In a fourth example of the method, optionally including one or more or each of the first through third examples, the method further comprises: pasting artificial intelligence (AI) prescribed landmarks, user-selected landmarks, coil setup, respiratory bellows, electrode setup, and other information on the privacy preserved camera image by matching in scale the landmarks using Affine transformation, and pasting on the privacy preserved camera image.

The disclosure also provides support for an imaging system, comprising: a camera configured to capture camera image data, a scanner configured to capture diagnostic imaging data, a table configured to receive an imaging subject, and a computing device having instructions stored on non-transitory memory and executable by a processor to: acquire camera image data of a field of view including the imaging subject and the table, via the camera, execute a table retention algorithm to remove camera image data other than image data of the imaging subject and the table from a camera image, execute a landmark detection algorithm configured to identify a privacy preserving region of the imaging subject, obscure camera image data of the camera image in the privacy preserving region to obfuscate personally-identifying image data and generate a privacy preserved camera image, register the privacy preserved camera image with the diagnostic imaging data captured by the scanner a camera-scanner coordinate transformation matrix to generate an accessible privacy preserved image that includes camera workflow information, obfuscated personally-identifying image data obfuscated, and is accessible via a standard medical imaging format, and store the accessible privacy preserved image in a memory of the computing device. In a first example of the system, the scanner is a computed tomography (CT) imaging system. In a second example of the system, optionally including the first example, the camera image data captured by the camera includes red, green, blue (RGB) image data and depth map image data.

As used herein, an element or step recited in the singular and preceded with the word “a” or “an” should be understood as not excluding plural of said elements or steps, unless such exclusion is explicitly stated. Furthermore, references to “one embodiment” of the disclosure do not exclude the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for a medical imaging system, comprising:
acquiring camera image data, including red, green, blue (RGB) image data and depth map image data, captured via a camera;
acquiring scanner image data captured via a scanner;
identifying a privacy region in the camera image data;
obscuring camera image data in the privacy region to generate a privacy preserved camera image;
registering the privacy preserved camera image with scanner image data, using a camera-scanner coordinate transformation matrix, to generate an accessible privacy preserved image that includes camera image data, scanner image data, obscured camera image data in the privacy region, and is accessible via a standard medical imaging format; and
storing the accessible privacy preserved image that can be viewed and navigated within a standard medical imaging format viewer.

2. The method of claim 1, wherein the privacy region in the camera image data is identified using a landmark detection algorithm.

3. The method of claim 2, wherein identifying the privacy region in the camera image data using the landmark detection algorithm includes identifying landmarks in RGB image data and/or depth map image data based on anatomy landmarks, and generating a bounding box using the anatomy landmarks.

4. The method of claim 3, further comprising using the bounding box to crop depth map information of the camera image data to generate the privacy region.

5. The method of claim 2, wherein the privacy region includes a face of a subject, and landmarks used by the landmark detection algorithm to identify the face includes ear landmarks to identify left and right bounds, and head to neck landmarks to identify superior and inferior bounds.

6. The method of claim 1, wherein identifying the privacy region comprises applying a table retention algorithm to the camera image data to remove camera image data from outside of a table region.

7. The method of claim 6, wherein applying the table retention algorithm comprises:
acquiring table dimensions of a table, including a thickness, a width, and a length of the table;
acquiring a depth ingress equal to a distance between a scanner bore ingress center of the scanner and the camera;
acquiring a bore ingress equal to a patient ingress bore diameter of the scanner;
generating a cylindrical region with a diameter that is equal to a sum of the thickness of the table and the bore ingress;
acquiring centerline region coordinates for the cylindrical region by applying the depth ingress to the depth map image data of the camera image data;
generating a cylinder mask having the diameter of the cylindrical region, the centerline region coordinates, and a main axis length that is equal to the length of the table; and
applying the cylinder mask to the RGB image data and depth map image data of camera image data to remove camera image data other than image data of the table and an imaging subject positioned on the table from the camera image data.

8. The method of claim 1, wherein camera image data in the privacy region is obscured via one or more of alpha blending, Poisson blending, or artificial intelligence (AI)-based blending.

9. The method of claim 1, wherein obscuring camera image data in the privacy region comprises blending RGB image data and depth map image data of the camera image data to remove identifiable features of an imaging subject in the privacy region.

10. The method of claim 1, wherein obscuring camera image data in the privacy region comprises blending depth map image data of the camera image data to remove identifiable features of an imaging subject in the privacy region.

11. The method of claim 1, wherein the standard medical imaging format is Digital Imaging and Communication in Medicine (DICOM®).

12. The method of claim 11, wherein adding additional layers of information comprises embedding burnt-in pixels into the privacy preserved camera image, the burnt-in pixels identifying user selected and/or AI-prescribed landmarks.

13. A method, comprising:
capturing camera image data, including RGB image data and depth map image data, via a camera;
capturing scanner image data via a scanner;
identifying a privacy region in the camera image data via a landmark detection algorithm;
applying a table-retention algorithm to the camera image data to remove camera image data from outside of a table region;
obscuring camera image data in the privacy region to generate a privacy preserved camera image;
adding additional layers of information from a scanning procedure to the privacy preserved camera image;
registering the privacy preserved camera image with scanner image data, using a camera-scanner coordinate transformation matrix, to generate an accessible privacy preserved image that includes camera image data, scanner image data, obscured camera image data in the privacy region, and is accessible via a standard medical imaging format; and
storing privacy preserved camera-based patient setup information as the accessible privacy preserved image that can be viewed and navigated within a standard medical imaging format viewer.

14. The method of claim 13, wherein obscuring the camera image data in the privacy region comprises fusing a foreground image and a background image using a first blending factor for the foreground image and a second blending factor for the background image, where the second blending factor is different from the first blending factor.

15. The method of claim 14, wherein the foreground image is the depth map image data and the background image is the RGB image data.

16. The method of claim 14, wherein fusing image data comprises:
using a map of facial anatomy to identify a bounding box for a face region;
matching in scale the bounding box of the face region and a digital body atlas;
pasting a transformed bounding box to the RGB image data using the first blending factor and the second blending factor.

17. The method of claim 13, further comprising pasting artificial intelligence (AI) prescribed landmarks, user-selected landmarks, coil setup, respiratory bellows, electrode setup, and other information on the privacy preserved camera image by matching in scale the landmarks using Affine transformation, and pasting on the privacy preserved camera image.

18. An imaging system, comprising:
a camera configured to capture camera image data;
a scanner configured to capture diagnostic imaging data;
a table configured to receive an imaging subject; and
a computing device having instructions stored on non-transitory memory and executable by a processor to:
acquire camera image data of a field of view including the imaging subject and the table, via the camera;
execute a table retention algorithm to remove camera image data other than image data of the imaging subject and the table from a camera image;
execute a landmark detection algorithm configured to identify a privacy preserving region of the imaging subject;
obscure camera image data of the camera image in the privacy preserving region to obfuscate personally-identifying image data and generate a privacy preserved camera image;
register the privacy preserved camera image with the diagnostic imaging data captured by the scanner a camera-scanner coordinate transformation matrix to generate an accessible privacy preserved image that includes camera workflow information, obfuscated personally-identifying image data obfuscated, and is accessible via a standard medical imaging format; and
store the accessible privacy preserved image in a memory of the computing device.

19. The imaging system of claim 18, wherein the scanner is a computed tomography (CT) imaging system.

20. The imaging system of claim 18, wherein the camera image data captured by the camera includes red, green, blue (RGB) image data and depth map image data.
